(19) United States
(12) Patent Application Publication
(10) Pub. No.: US 2026/0240876 A1
(43) Pub. Date: Aug. 20, 2026

(54) TREATMENT OF SLEEP APNEA WITH CBD

(71) Applicant: Harmony Biosciences Management, Inc., Plymouth Meeting, PA (US)

(72) Inventors: Joseph Palumbo, Saint Davids, PA (US); Donna Gutterman, Raleigh, NC (US); Terri Sebree, Gladwyne, PA (US)

(73) Assignee: Harmony Biosciences Management, Inc., Plymouth Meeting, PA (US)

(21) Appl. No.: 19/641,271

(22) Filed: Apr. 7, 2026

Related U.S. Application Data
(63) Continuation of application No. 18/263,092, filed on Jan. 28, 2022, now abandoned.
(60) Provisional application No. 63/142,835, filed on Jan. 28, 2021.

(51) Int. Cl. A61K 31/00; A61M 21/00

(52) U.S. Cl. CPC A61K 31/658; A61M 21/00

(57) ABSTRACT

Provided are methods of treating sleep disorders in patients by transdermally administering an effective amount of cannabidiol (CBD) to a subject in need thereof.

### CROSS-REFERENCE TO RELATED APPLICATIONS

This application is a continuation of U.S. patent application Ser. No. 18/263,092, filed Jul. 26, 2023, which is a 371 National Phase Application of International Application No. PCT/IB2022/050781, filed Jan. 28, 2022, which claims benefit and priority to U.S. Provisional Application No. 63/142,835, filed Jan. 28, 2021. The contents of each are incorporated herein by reference in their entirety.

### FIELD OF THE TECHNOLOGY

The present disclosure relates to methods of treating sleep disorders in patients by transdermally administering an effective amount of cannabidiol (CBD) to a subject in need thereof.

### BACKGROUND

Developmental and epileptic encephalopathies (DEEs) are a severe group of neurodevelopmental disorders characterized by seizures and abnormal electroencephalogram activity that negatively impact development. There exists a need for a safe and effective treatment of refractory seizures, e.g., in children with DEE. This can be particularly difficult to treat when the subject has a co-morbidity such as Autism Spectrum Disorder (ASD).

Sleep disturbances are a common problem associated with DEE, ASD and DEE-ASD children. Sleep breathing disorders, such as sleep apnea, are one type of sleep disturbance that is seen in patients with DEE, ASD, and DEE-ASD. In addition to the need for treatment of epilepsy in these patients, it is also desirable to improve the sleep patterns of such patients.

Sleep disturbances are not limited to patients with DEE, ASD, or DEE-ASD, as many adults also suffer from sleep disturbances. Treatments that improve sleep disturbances would be useful for an extensive population.

### SUMMARY

EPIDIOLEX oral CBD solution has been approved for treatment of epilepsy in children with Lennox-Gastaut and Dravet syndrome. However, oral delivery has translated to gastrointestinal (GI) adverse events, e.g., the EPIDIOLEX label reports somnolence and sedation in 32% of its patients and was dose related. EPIDIOLEX Cannabidiol Oral Solution Label (June 2018). Oral CBD also has the potential to degrade in gastric acid into THC, which can be associated with unwanted psychoactive effects. Id.

There exists a need for an efficacious treatment for specific targeted (refractory) seizure types in children diagnosed with autism spectrum disorders (e.g., treatment of refractory epilepsy in autism spectrum disorder). Quality of life (QoL) of children with drug-resistant epilepsy declines with greater number of ASMs, greater seizure frequency, and lower IQ, further underscoring a need for new therapies. A treatment is also needed that does not produce unwanted side effects such as somnolence, lethargy, withdrawal, or sedation.

The present disclosure relates to a method of treating refractory seizures in a subject, including transdermally administering an effective amount of CBD to the subject. The CBD can be ZYN002, which is a transdermal synthetic CBD gel formulation. The subject can be a child, including a child having a disease such as one or more of autism spectrum disorder (ASD), developmental and epileptic encephalopathy (DEE), or epilepsy. For example, the subject can be children with co-morbid ASD with epilepsy. Effective treatment also includes related improvements in sleep quality, sleep onset, and sleep maintenance in children.

In some examples, the present disclosure relates to a method of treating a human suffering from an autism spectrum disorder (ASD). The method can include administering an effective amount of cannabidiol (CBD) to the human in need thereof to effectively treat a refractory seizure type in humans with ASD.

In some examples the refractory seizure type is focal impaired awareness seizures (FIAS), generalized tonic-clonic seizures (GTCS), or focal to bilateral tonic-clonic seizures (FBTCS).

In some examples, the CBD can be administered transdermally. The effective amount of CBD can range from about 250 mg to about 1000 mg total daily. The effective amount of CBD can be 250 mg total daily. The effective amount of CBD can be 500 mg total daily. The effective amount of CBD can be 750 mg total daily. The effective amount of CBD can be 1000 mg total daily. The CBD can be administered in a single daily dose. The CBD can be administered in two daily doses.

In some examples, the treatment includes an improvement in sleep-related impairment. An improvement in sleep-related impairment includes improvements in sleep quality, sleep onset, total sleep, initiating and maintain sleep, sleep wake transition, or disorders of arousal and nightmares.

Treatment of the patient with CBD, in some embodiments, will reduce the occurrence of sleep breathing disorders (SBD) such as sleep apnea. The use of CBD may reduce the incidence of sleep apneas and other sleep disorders in patients with DEE, ASD, or DEE-ASD. The use of CBD may also improve sleep breathing disorders in adults and children that do not have DEE, ASD or DEE-ASD.

In some examples, the CBD is a synthetic CBD. The CBD can be a pure CBD. The CBD can be botanically derived. The cannabidiol can be (−)-cannabidiol.

In some examples, the CBD is formulated as a gel. The CBD can be formulated as a permeation-enhanced gel.

In some examples, upon beginning administering the effective amount of CBD, circadian rhythm is maintained or improved. In some examples, administering the effective amount of CBD does not become addictive and the human subjects experience no excessive somnolence.

The present disclosure relates to a method of treating refractory seizures in a human suffering from autism spectrum disorder (ASD). The method includes administering an effective amount of cannabidiol (CBD) to the human in need thereof to effectively treat a refractory seizure type in humans with ASD. The human subjects can include children diagnosed with ASD. The human subjects can also include human subjects having comorbidities of ASD and refractory epilepsy.

The present disclosure relates to a method of treating refractory seizures in a child suffering from developmental and epileptic encephalopathies (DEE). The method can include administering an effective amount of cannabidiol (CBD) to the child in need thereof to effectively treat a refractory seizure type in children with DEE.

The present disclosure relates to a method of treating sleep disturbances in a human subject. The method can include administering an effective amount of cannabidiol (CBD) to the human in need thereof to treat the sleep disturbances. In an embodiment, the sleep disturbances are caused by episodes of sleep apnea.

Sleep disturbances can be treated by transdermal CBD administration. The effective amount of CBD ranges from about 250 mg to about 1000 mg total daily. The effective dosage of CBD is in the range of 10-25 mg/kg per dose. The CBD can be administered in a single daily dose or can be administered in two daily doses. In an embodiment, the CBD is pure CBD. The CBD can be synthetic CBD or botanically derived CBD. In an embodiment, the CBD is (−)-cannabidiol. In an embodiment, the CBD is formulated as a gel. The CBD gel can be formulated as a permeation-enhanced gel.

Use of CBD to treat sleep disturbances results in an improvement in sleep-related impairment. An improvement in sleep-related impairment includes improvements in sleep quality, sleep onset, total sleep, initiating and maintain sleep, sleep wake transition, or disorders of arousal and nightmares.

In an embodiment, the subject having sleep disturbances suffers from obstructive sleep apnea. The obstructive sleep apnea is associated with obesity or an overweight condition of the subject. In an embodiment, the subject has a BMI of greater than 25. In an embodiment, the subject is an adult. Alternatively, or in addition, the subject can be between 3 and 18 years old. In some embodiments, the subject may also have ASD or DEE-ASD. In an embodiment, upon beginning administering the effective amount of CBD, circadian rhythm is maintained or improved.

### BRIEF DESCRIPTION OF THE DRAWINGS

FIG. 1 displays a study design and treatment for cannabidiol transdermal gel, in accordance with the present disclosure.

FIG. 2 displays the scoring for the good day/bad day assessment.

FIG. 3 is a graph indicating the efficacy of the treatment period.

FIG. 4 is a graph indicating the percentage of patients with reduction in seizures.

FIG. 5 is a graph indicating median percentage reduction from baseline in 28-day frequency of seizures.

FIG. 6 is a graph indicating the percentage of patients with reduction in seizures with co-morbid autism spectrum disorder (ASD) at baseline.

FIG. 7 displays the percentage of patients (DEE) above threshold for clinically significant sleep problems at Baseline and Week 26 of ZYN002 treatment based on the Sleep Disturbance Scale for Children (SDSC).

FIG. 8 displays the percentage of patients (DEE) with a threshold t-score >70 at Baseline and Week 26, corresponding to clinically significant sleep problems.

FIG. 9 displays the percentage of patients (DEE) with co-morbid ASD above threshold for clinically significant sleep problems at Baseline and Week 26 of ZYN002 treatment based on the Sleep Disturbance Scale for Children (SDSC).

FIG. 10 shows the percentage of patients (DEE) with co-morbid ASD with a threshold t-score >70 at Baseline and Week 26, corresponding to clinically significant sleep problems.

FIG. 11 displays a distribution of Good Day/Bad Day ratings at Baseline and Month 6 of ZYN002 treatment.

### DETAILED DESCRIPTION

Provided herein is a method of treating seizures, such as refractory type seizures, in a subject by transdermally administering an effective amount of CBD to the subject. The subject can have ASD as well as epilepsy (DEE). Also provided herein is a method of treating sleep disorders (e.g., sleep apnea) in any human subject.

The trial summarized in the examples—a Phase 2 open label clinical trial—assessed the safety, tolerability, and efficacy of transdermal administration of CBD in DEE, a heterogeneous group of rare pediatric epilepsy syndromes, including but not limited to Dravet Syndrome (DS), Lennox-Gastaut Syndrome (LGS), and West syndrome.

Fourteen of the children in the DEE study were prospectively identified as being comorbidly diagnosed with ASD. Although there may be a perception that seizures in the general population of ASD may be more easily medically managed, all of the children in the DEE study had difficult to treat and/or refractory epilepsy—by extension, refractory epilepsy in ASD is also a rare population and can also function as a therapeutic indication. Refractory epilepsy is resistant to drugs, and treatment of refractory epilepsy fails to provide substantial seizure freedom. Similarly, refractory seizures are resistant to drugs, and treatment of refractory seizures fails to provide substantial seizure freedom.

Of the 14 ASD children with DEE, 11 showed overall evidence of positive treatment benefit on total seizure number, and 11 patients were diagnosed with seizure types of focal impaired awareness seizures (FIAS) or tonic-clonic seizures (TCS), including generalized tonic-clonic seizures (GTCS) and focal to bilateral tonic-clonic seizures (FBTCS). Of the 11 ASD children with FIAS or TCS, 8 children showed seizure improvement during open-label treatment with ZYN002 as an adjunct to stable standard of care.

Transdermally administering an effective amount of CBD can have a positive impact on sleep in children with DEE, e.g., children with co-morbid ASD with epilepsy (DEE). For example, transdermally administering CBD can be used for treatment of disorders of the initiation and maintenance of sleep (DIMS) in ASD with epilepsy. Transdermally administering an effective amount of CBD can increase the number of good days and decrease the number of bad days experienced by a child patient.

### Definitions

As used herein, the term “treating” or “treatment” refers to mitigating, improving, relieving, or alleviating at least one symptom (such as a behavioral symptom) of a condition, disease or disorder in a subject, such as a human, or the improvement of an ascertainable measurement associated with a condition, disease or disorder.

As used herein, the terms “clinical efficacy”, “clinically effective”, and the like refer to efficacy as demonstrated in a clinical trial conducted by the Food and Drug Administration (FDA), or any foreign counterpart, e.g., the European Medicines Agency (EMA).

As used herein, the term “cannabidiol” or “CBD” refers to cannabidiol; cannabidiol prodrugs; pharmaceutically acceptable derivatives of cannabidiol, including pharmaceutically acceptable salts of cannabidiol, cannabidiol prodrugs, and cannabidiol derivatives. CBD includes, 2-[3-methyl-6-(1-methylethenyl)-2-cyclohexen-1-yl]-5-pentyl-1,3-benzenediol as well as to pharmaceutically acceptable salts, solvates, metabolites (e.g., cutaneous metabolites), and metabolic precursors thereof. The synthesis of CBD is described, for example, in Petilka et al., Helv. Chim. Acta, 52:1102 (1969) and in Mechoulam et al., J. Am. Chem. Soc., 87:3273 (1965), which are hereby incorporated by reference.

ZYN002 is a pharmaceutically manufactured transdermal CBD gel for reduction of seizures in patients and improvement in behavioral symptoms in patients. The patients can have ASD, epilepsy, DEE, Fragile X Syndrome (FXS), along with other diseases. Some of the patients may have comorbidities with multiple diseases, such as ASD with epilepsy. In some examples, the human subjects with ASD includes children diagnosed with ASD, and the human subjects can have comorbidities of ASD and epilepsy, such as refractory epilepsy. In some examples, the terms “child” or “children” refer to patients that are from 3 to 18 years of age.

The term “epileptic encephalopathy” refers to epileptic activity that itself contributes to severe cognitive and behavioral problems above and beyond what may be expected from the underlying pathology alone (e.g., cortical malformation). Onset of these impairments can occur at any age.

The term “developmental and epileptic encephalopathy” or “DEE” refers to severe epilepsy disorders with onset in infancy and childhood. DEE is characterized by the presence of multiple focal and generalized seizure types and severe cognitive and behavioral problems. In DEE, cognitive and behavioral problems can occur independently of seizure activity, even before seizures become frequent, suggesting a developmental component in addition to an epileptic component to DEE. Such impairment can happen early, or worsen over time. Scheffer, “ILAE classification of the epilepsies: Position paper of the ILAE Commission for Classification and Terminology” Epilepsia 58(4):512-521, 2017. DEE includes genetic epilepsies, such as CDKL5, SCNIA-, and STXBP1-related disorders. It also includes Lennox-Gastaut Syndrom (LGS), Ohtahara, West, Landau-Kleffner, Doose, Dravet Syndrome (DS), and Infantile Spasms (IS). DEEs with onset ≤18 months have an incidence of 1 in 2000 live births.

As used herein, the term “transdermally administering” refers to contacting the CBD with the patient's or subject's skin under conditions effective for the CBD to penetrate the skin.

The term developmental and epileptic encephalopathy (DEE) was introduced by the International League Against Epilepsy (ILAE) Task Force on Classification and Terminology (Scheffer et al. 2017) to more fully describe the clinical presentation of co-existing developmental impairment and epileptic encephalopathy. Historically, epileptic encephalopathy, without the term ‘developmental,’ was used in the broader sense to encompass both concepts. In 2001, ILAE recognized epileptic encephalopathies as a distinct category. Engel, “A proposed diagnostic scheme for people with epileptic seizures and with epilepsy: report of the ILAE Task Force on Classification and Terminology” Epilepsia 42:796-803 (2001). The ILAE defined an epileptic encephalopathy as a condition in which “the epileptiform EEG abnormalities themselves are believed to contribute to a progressive disturbance in cerebral function.” In 2010, the ILAE redefined epileptic encephalopathy as a condition where the epileptic activity itself may contribute to severe cognitive and behavioral problems above and beyond what might be expected from the underlying pathology alone (e.g., cortical malformation), and that these can worsen over time. Berg et al., “Revised terminology and concepts for organization of seizures and epilepsies: report of the ILAE Commission on Classification and Terminology, 2005-2009” Epilepsia 51:676-685 (2010).

The change to include ‘developmental’ in the description was done to allow specific recognition of patients who present with both developmental impairment and epileptic encephalopathy verses developmental impairment without frequent epileptic activity associated with developmental impairment or epileptic encephalopathy where there is no pre-existing development of impairment. A key component of the concept is that amelioration of the epileptiform activity may have the potential to improve the developmental consequences of the disorder (Scheffer et al. 2017).

The overall incidence and prevalence of developmental epileptic encephalopathies are low. Patients with DEE may include, but are not limited to, patients with Lennox-Gastaut syndrome, Dravet syndrome, Doose syndrome (Epilepsy with Myoclonic Atonic Seizures (EMAS)), West syndrome (Infantile Spasms), Landau-Kleffner syndrome, or genetic disorders such as CDKL5 encephalopathy and CHD2 encephalopathy.

Seizures are generally refractory to antiseizure medications (ASMs), such as standard antiepileptic drugs (AEDs). For example, the seizures in patients with DEE are generally refractory to AEDs. As a result, more aggressive adjunctive use of AEDs considered effective in suppressing interictal epileptiform discharges (e.g., benzodiazepines, valproic acid, and lamotrigine), immunomodulatory therapies (e.g., corticosteroids, intravenous immunoglobulin [IVIG], plasmapheresis), ketogenic diet, and surgical options are often considered. Additionally, oral administration of AEDs can be difficult due to behavioral and cognitive impairments.

While patients with DEE may present with a variety of seizure types and sub-disorders, the only DEE subtypes for which one or more AEDs are currently approved by the US FDA for adjunctive therapy are Lennox-Gastaut Syndrome, Dravet Syndrome, and infantile spasms (Table 1). Children with DEE can be medically fragile and have multiple comorbidities including motor and cognitive impairments, ASD, and sleep disturbance, which further increase disability. In some examples, children are defined as 3 to less than 18 years of age.

TABLE 1
Current US FDA Approved Medicines by DEE Subtype

| DEE Subtype | US FDA Approved Medicines |
|---|---|
| Lennox-Gastaut syndrome | Cannabidiol, clobazam, lamotrigine, |
| | rufinamide, topiramate, felbamate, |
| | clonazepam |
| Dravet Syndrome | Cannabidiol |
| Infantile spasms | Vigabatrin, adrenocorticotropic |
| (West Syndrome) | hormone (ACTH) |

Given treatment refractoriness and limited approved medicines with evidence from controlled trials, clinicians are often left with using standard AEDs in a trial and error fashion, largely based on clinical experience or open label trials. Lennox-Gastaut syndrome and Dravet syndrome have been the DEE subtypes for which the most evidence from controlled trials of antiepileptic drugs has been generated.

Similar to DEE, children with ASD have refractory seizure types. For example, there exists a need for treatment of refractory epilepsy in ASD.

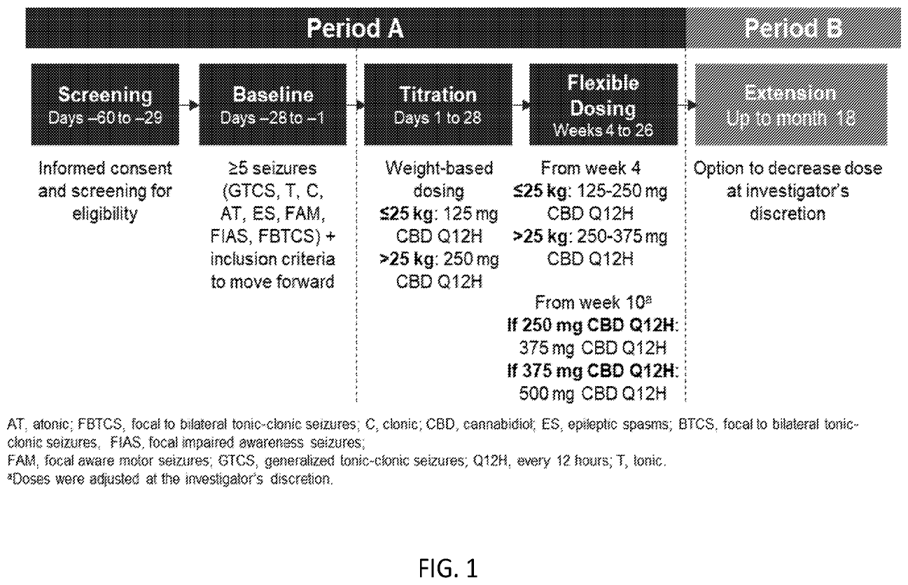

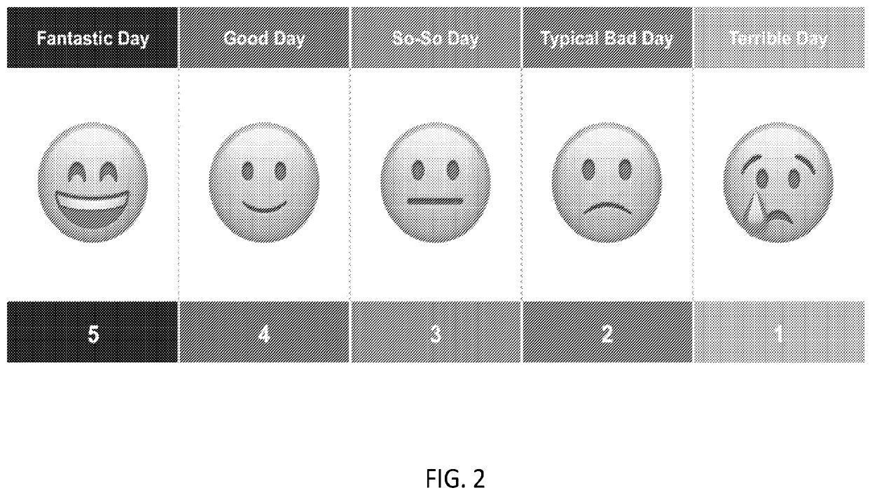

Table 2 displays the descriptions related to the QoL assessments for the BELIEVE study (FIG. 1, discussed further below, displays the study design and treatment for the BELIEVE study). QOL assessments were caregiver-rated and included the ELDQOL scale, a daily “good day/bad day” assessment, and qualitative feedback (Table 2 and FIG. 2).

TABLE 2
QoL Assessments

| Assessment | Description |
|---|---|
| ELDQOL^{a} | Scale is a questionnaire measuring QoL over the |
| | prior 4 weeks |
| | Subscales include seizure severity, seizure-related |
| | injuries, AED side-effects, behavior, mood, physical, |
| | cognitive and social functioning, parental concern, |
| | communication, overall QOL and overall health |
| | Higher subscale scores indicate poorer QoL |
| Daily good | Daily assessment of a patient's QoL, in which |
| day/bad day | parents were asked to provide an overall daily score, |
| assessment | considering factors such as seizure frequency, |
| | alertness, behavior, mood, etc. |
| | Scoring is described in FIG. 2 |
| Qualitative | Qualitative assessment of improvement, worsening, |
| caregiver | or no change for the patient and family, such as but |
| feedback | not limited to, daily activities, school attendance and |
| | alertness |
| | Investigators asked the following questions of |
| | caregivers at week 26: |
| | Has anything improved for “X” and your family |
| | since “X” has been using the gel? |
| | Has anything got worse for “X” and your family |
| | since “X” has been using the gel? |
| | Let me just ask about a few specific things: |
| | Daily activities, e.g., school attendance? If so how? |
| | Alertness? If so how? |
| | Two independent evaluators working separately |
| | coded caregiver statements using ATLAS.ti and |
| | classified statements into domains |

ELDQOL: Epilepsy and Learning Disabilities Quality of Life; QoL: quality of life.
^{a}The ELDQOL was modified with written permission from the developers; modifications did not impact the validity of the questionnaire.

Table 3 displays description for the Sleep Disturbance Scale for Children (SDSC), the assessment that was conducted by caregivers to evaluate sleep disorders in children.

TABLE 3
Sleep Assessment

| Assessment | Description |
|---|---|
| SDSC | A 26-item, Likert-type scale (5 possible answers per item) to evaluate |
| | sleep disorders in children, and to provide an overall measure of sleep |
| | disturbance suitable for use in clinical screening and research |
| | The items are organized into 6 categories: disorders of initiating or |
| | maintaining sleep (DIMS, 7 items), sleep breathing disorders (SBD, 3 |
| | items), disorder of arousal/nightmares (DA, 3 items), sleep wake |
| | transition disorder (SWTD, 6 items), disorders of excessive somnolence |
| | (DOES, 5 items), and sleep hyperhidrosis (SHY, 2 items) |
| | Patients/parents use a five-point scale to indicate frequency from 1 |
| | (never) to 5 (always). Higher scores indicate more acute sleep |
| | disturbances. Scores are tallied for each of the six sleep-disorder |
| | categories, and an overall score is calculated. |

Reductions in sleep disturbances, for example by reducing sleep apnea, is believed to be related to both improved night time sleep (e.g., sleep transitions and sleep maintenance) as well as with better daytime alertness because of better night time sleep. Better sleep in individuals, particularly those having DEE, ASD, or DEE-ASD, can lead to better daytime function, better daytime cognition, and better educational performance.

### Transdermal Pharmaceutical Compositions

Transdermal delivery of cannabinoids (e.g., CBD) has benefits over oral dosing because it allows the drug to be absorbed through the skin directly into the bloodstream. This avoids first-pass liver metabolism, enabling lower dosage levels of active pharmaceutical ingredients with a higher bioavailability and improved safety profile. Transdermal delivery also avoids the gastrointestinal tract, lessening the opportunity for GI related adverse events and the potential degradation of CBD by gastric acid into THC, which can be associated with unwanted psychoactive effects. Moreover, transdermal delivery of CBD reduces the intensity and frequency of somnolence adverse events, which are typically present in oral dosing of CBD. Transdermal delivery of CBD can avoid liver function adverse events, which are typically present in oral dosing of CBD. In some embodiments, transdermally administering an effective amount of CBD reduces an intensity of at least one adverse event by about 15% to about 95% relative to orally administering CBD. Exemplary transdermal cannabinoid delivery systems are described in U.S. Pat. Nos. 8,435,556 and 8,449,908, both of which are incorporated herein by reference.

The CBD can be in a gel form and can be pharmaceutically-produced as a clear gel, such as a permeation-enhanced gel that is designed to provide controlled drug delivery transdermally with once- or twice-daily dosing. The CBD gel can between 1% (wt/wt) CBD to 7.5% (wt/wt) CBD. The CBD gel can have, for example, 4.2% (wt/wt) CBD or 7.5% (wt/wt) CBD). The CBD gel can be applied topically by the patient or caregiver to the patient's upper arm and shoulder, back, thigh, or any combination thereof.

The CBD gel can include diluents and carriers as well as other conventional excipients, such as wetting agents, preservatives, and suspending and dispersing agents. The CBD can be a synthetic CBD. The CBD can be a pure CBD. The CBD can be botanically derived. The CBD can be (−)-cannabidiol.

The transdermal preparation can be a cream, a salve, a lotion, or an ointment. The CBD can be delivered by a bandage, pad or patch.

The CBD can be administered transdermally on the subject's upper arm and shoulder. In some embodiments, the CBD is administered transdermally on the subject's thigh or back.

The CBD gel can include a solubilizing agent, a permeation enhancer, a solubilizer, antioxidant, bulking agent, thickening agent, and/or a pH modifier. The composition of the CBD gel can be, for example, a. cannabidiol present in an amount of about 0.1% to about 20% (wt/wt) of the composition; b. a lower alcohol having between 1 and 6 carbon atoms present in an amount of about 15% to about 95% (wt/wt) of the composition; c. a first penetration enhancer present in an amount of about 0.1% to about 20% (wt/wt) of the composition; and d. water in a quantity sufficient for the composition to total 100% (wt/wt). Other formulations of the CBD gel can be found in International Publication No. WO 2010/127033, the entire contents of which are incorporated herein by reference.

In some examples, there may be other ways to provide the CBD gel, e.g., via a pump or as an alternative transdermal formulation.

The effective amount of CBD can range from about 250 mg to about 1000 mg total daily, which can be administered in a single daily dose or twice daily dosing. For example, the effective amount of CBD can be about 250, 500, 750, or 1000 mg total daily, which can be administered in a single daily dose or in two daily doses. To illustrate, the 1000 mg total daily dose can be administered in two daily doses of 500 mg.

In some examples, upon beginning administering the effective amount of CBD, circadian rhythm is maintained or improved. Administering the effective amount of CBD does not become addictive for at least some of the patients, if not all of the patients. Besides the benefits of not developing an addiction upon beginning administering the effective amount of CBD, the human subjects, e.g., the patients, can experience no excessive somnolence.

### Sleep Apnea

Sleep apnea is a sleep disorder characterized by having one or more pauses in breathing or shallow breaths during sleep. Each pause in breathing, called an apnea, can last from a few seconds to minutes, and may occur 5 to 30 times or more an hour. For moderate to severe sleep apnea, the most common treatment is the use of constant positive airway pressure (CPAP), which helps to maintain an open airway during sleep by means of a constant flow of pressurized air into the patient's mouth and/or nose. The patient typically wears a mask that covers the nose and/or mouth and which is connected by a flexible tube to a small bedside compressor. This type of treatment, while effective, can be uncomfortable.

There is some correlation between obstructive sleep apnea (OSA) and weight. The prevalence of OSA in obese or severely obese patients is nearly twice that of normal-weight adults. Furthermore, patients with mild OSA who gain 10% of their baseline weight are at a sixfold-increased risk of progression of OSA, and an equivalent weight loss can result in a more than 20% improvement in OSA severity (Peppard et al., Longitudinal study of moderate weight change and sleep-disordered breathing. JAMA. 2000; 284(23):3015-3021). Moreover, the higher prevalence of OSA in obese subjects is not limited to adults; recent data show that obese children have a 46% prevalence of OSA when compared with children seen in a general pediatric clinic (33%) (Rudnick et al. Prevalence and ethnicity of sleep-disordered breathing and obesity in children. Otolaryngol Head Neck Surg. 2007; 137(6):878-882).

Fat deposition at specific sites may have an effect on sleep apnea. Fat deposition in the tissues surrounding the upper airway appears to result in smaller passageways and increased collapsibility of the upper airway, predisposing the person to apnea. Fat deposits around the thorax (truncal obesity) reduce chest compliance and functional residual capacity, and may increase oxygen demand. (Romero Corral et al. Interactions Between Obesity and Obstructive Sleep Apnea Chest. 2010 March; 137(3): 711-719).

While excessive weight is one of the leading causes of sleep apnea, there are other causes of sleep apnea that are unrelated to weight. Some patients may have pre-existing anatomical or neurological conditions that can cause sleep apnea. For example, central sleep apnea is a disorder in which an individual's breathing repeatedly stops and starts during sleep due to the brain not sending proper signals to the muscles that control your breathing during sleep. People with thicker necks or a narrow throat will have narrower airways, which can lead to sleep apnea. Tonsils or adenoids also can enlarge and block the airway, particularly in children. All of these conditions can cause sleep apnea episodes in individuals regardless of weight or BMI.

In an embodiment, sleep disturbances in a human subject are treated by administering an effective amount of cannabidiol (CBD) to the human in need thereof to treat the sleep disturbances. In some embodiments, the sleep disturbances are caused by episodes of sleep apnea. In an embodiment, the CBD is administered transdermally as has been described previously. Typical doses of transdermal CBD range from about 10 mg/kg to about 25 mg/kg. In some embodiments, the effective amount of CBD ranges from about 250 mg to about 1000 mg total daily. CBD can be administered in a single daily dose or two daily doses. CBD, in some embodiments, is administered at least 8 hours, at least 6 hours, or at least 4 hours before the expected bed time of the patient. CBD used for treatment of sleep disturbances can be administered between about 9 am and 4 μm, between 10 am and 3 μm, or between 11 am and 2 pm.

In an embodiment, the subject having sleep disturbances suffers from obstructive sleep apnea. The obstructive sleep apnea is associated with obesity or an overweight condition of the subject. In an embodiment, the subject has a BMI of greater than 25. In an embodiment, the subject is an adult. Alternatively, or in addition, the subject can be between 3 and 18 years old. In some embodiments, the subject may also have ASD or DEE-ASD. In an embodiment, upon beginning administering the effective amount of CBD, circadian rhythm is maintained or improved.

### EXEMPLIFICATION

### Example 1: Study of ZYN-002 in Children with DEE

This was a sequential, multi-stage, open-label, multi-national, multiple-center, multiple-dose study to assess the long-term safety, tolerability, and efficacy of ZYN002 (transdermal CBD gel) in child and adolescent epilepsy patients from 3 to 18 years of age having seizures associated with developmental and epileptic encephalopathies (DEE) according to the International League Against Epilepsy (ILEA) classification (Scheffer et al. 2017). The study was conducted to evaluate efficacy of ZYN002 in DEE patients with ASD. The objective of the study also included evaluating the effects of ZYN002 transdermal CBD gel on QoL, sleep, and caregiver qualitative assessment in child and adolescent patients with DEEs.

FIG. 1 displays a study design and treatment of Example 1 for cannabidiol transdermal gel in BELIEVE (ZYN2-CL-025), in accordance with the present disclosure. ZYN002 was administered in total daily doses of 250 mg to 1000 mg over an initial 26-week treatment period (Period A) followed by an up to 46-week extension (Period B). Results for the Period A and through Month 12 of Period B and safety results through 72 weeks are discussed herein.

Approximately 48 patients enrolled Period A with 40 patients progressing to completing open-label treatment in Periods A. In Period A, patients underwent a baseline period of 4-weeks, followed by a 4-week titration period, and a 22-week flexible dosing maintenance period. Patients were treated for a total of 26 weeks in Period A.

Approximately 29 patients entered Period B with 28 patients progressing to completion. One patient withdraw consent at Week 42. In Period B, patients continued to receive ZYN002 for up to an additional 46 weeks at the same maintenance dose they were receiving at Week 26 (e.g., end of Period A).

Patients received twice daily applications (every 12 hours±2 hours) of study drug for the 26-week treatment period and the 46-week extension period (72-week total treatment duration).

Enrolled patients received weight-based initial doses of 250 mg daily or 500 mg daily of ZYN-002. Patients weighing ≤25 kg could be titrated up to 750 mg daily and patients weighing >25 kg could be titrated up to 1,000 mg daily.

### Diagnosis and Criteria for Inclusion

Patients participating in this study had a diagnosis of developmental and epileptic encephalopathy. Patients were male and female, between 3 and 18 years of age, and had a body mass index between 13 and 35 kg/m², and weighed no less than 12 kg.

Patients had a diagnosis of developmental and epileptic encephalopathy (DEE) as defined by the International League Against Epilepsy Classification (Scheffer 2017) with generalized motor (i.e. generalized tonic-clonic, tonic, clonic, atonic, epileptic spasms), focal aware motor, focal impaired awareness or focal to bilateral tonic-clonic seizures. Examples of DEE that were enrolled included, but were not limited to: Lennox-Gastaut Syndrome, Dravet Syndrome, West Syndrome/Infantile Spasms and Doose Syndrome. The diagnosis must have been established for ≥1 years and documented by history and examination and review of appropriate studies, which included electroencephalogram (EEG), magnetic resonance imaging (MRI) scan, or genetic testing.

A stable regimen of 1 to 4 ASMs for the patients was maintained from the baseline period throughout the entire study.

Patients had experienced five or more seizures of the following type(s) in total during the baseline period: generalized motor (i.e. generalized tonic-clonic, tonic, clonic, atonic or epileptic spasms), focal aware motor, focal impaired awareness or focal to bilateral tonic-clonic seizures. A cluster of epileptic spasms was counted as a single seizure.

Patients had a history of developmental delay with regression, slowing or plateau in at least one developmental domain after seizure onset as determined by the Investigator.

### Criteria for Exclusion

Patients were excluded for the study, including for the following reasons: use of tetrahydrocannabinol or CBD product ≤12 weeks before screening; treatment with a strong inhibitor/inducer of CYP3A4; change in ASM regimen or epilepsy dietary therapy within the previous 4 weeks; or alanine aminotransferase, aspartate aminotransferase, or total bilirubin levels ≥3× the upper limit of normal (ULN).

### Application Sites

Approved application sites for the gel were the right and left upper arm as specified in Table 4.

TABLE 4
Dosing Application

| CBD | | | |
|---|---|---|---|
| Daily | # of | # Sachets | |
| Dose | Sachets in | in | Application Site |
| (mg) | Morning | Evening | Q12H (+/−2 hrs) |
| 250 | 1 | 1 | 1 sachet to right or left upper |
| | | | arm/shoulder |
| 500 | 2 | 2 | 1 sachet to each upper right and |
| | | | left upper arm shoulder |
| 750 | 3 | 3 | 2 sachets to either the right or left |
| | | | upper arm/shoulder and 1 sachet |
| | | | to the opposite arm/shoulder. |
| 1000 | 4 | 4 | 2 sachets to each right and |
| | | | left shoulder |

If redness occurred at the application sites, after consultation with Investigator, ZYN002 was temporarily applied to the right and left upper thighs. Patients with low BMIs and/or small arms were allowed to have ZYN002 applied to the upper right or left thighs. Sequence of application was 1 sachet to each upper left and right arm/shoulder and 1 sachet to each right and left upper thigh.

If applied to the right and/or left upper thighs the procedure was the same as described for the left and right upper arms/shoulders. Parents/caregivers applying the gel wore gloves. The parent/caregiver assured that the gel was rubbed in completely, no gel was remaining on the gloves, and the skin surface where the gel was applied was no longer shiny and dry to the touch prior to dressing. Parents/caregivers were able to apply an approved moisturizing lotion, 2 hours following dosing.

### Product, Dosage and Mode of Administration

The product was ZYN002 (Cannabidiol: CBD), 4.2% gel, topical. And the drug was supplied as sachets containing 2.98 g of gel to deliver 125 mg of CBD/sachet. It was applied by using one (1) to four (4) sachets in the morning and evening to achieve the appropriate total daily dose for each patient based upon the treatment group.

The Treatments were as follows:
Treatment A—125 mg CBD Q12H (±2 hours); for a total daily dose 250 mg CBD (1 sachet in morning and 1 sachet in evening).
Treatment B—250 mg CBD Q12H (±2 hours); for a total daily dose of 500 mg CBD (2 sachets in morning and 2 sachets in evening).
Treatment C—375 mg CBD Q12H (±2 hours); for a total daily dose of 750 mg CBD (3 sachets in morning and 3 sachets in evening).
Treatment D—500 mg CBD Q12H (±2 hours); for a total daily dose of 1000 mg CBD (4 sachets in morning and 4 sachets in evening).

### Period A: Baseline Period

During the 4-week Baseline Period, parents and/or caregivers recorded the number of seizures of the following types in a seizure diary:
Generalized tonic-clonic (“primary generalized tonic-clonic’) seizures
Focal impaired awareness seizures
Focal to bilateral tonic-clonic seizures
Focal aware seizures with motor signs
Tonic seizures
Clonic seizures
Atonic seizures
Epileptic spasms (A cluster of epileptic spasms was counted as a single seizure.)

Seizures of the following types were captured in the daily diary at the same time and for the same duration every day, as determined by the investigator (e.g. 6:00 PM for 10 minutes):
Myoclonic seizures
Absence seizures
Focal aware seizures without motor signs (e.g. focal sensory seizures)

### Period A: Titration Period

The initial dose for patients <25 kg was 125 mg CBD Q12H (±2 hours), for a total daily dose of 250 mg CBD for the four-week titration period. At the week four visit (Visit 4), based on Investigator discretion, the dose could remain at 250 mg CBD daily or be increased to 250 mg CBD Q12H (±2 hours), for a total daily dose of 500 mg CBD (4 sachets) for the remaining 22 weeks of the treatment period.

Patients weighing >25 kg received 250 mg CBD Q12H (±2 hours), for a total daily dose of 500 mg CBD for the four-week titration period. At the week four visit (Visit 4), based on Investigator discretion, the dose could remain at 500 mg CBD daily or be increased to 375 mg CBD Q12H (±2 hours), for a total daily dose 750 mg CBD (6 sachets) for the remaining 22 weeks of the treatment period.

### Period A: Maintenance Period

At Week 10, patients taking 500 mg CBD daily could be increased to 750 mg CBD daily (6 sachets) and patients taking 750 mg CBD daily could be increased to 1000 mg CBD (8 sachets).

The Investigator decreased the dose as needed based on safety and tolerability after the patient started the maintenance period. Patients taking CBD 250 mg Q12H (±2 hours); total daily dose 500 mg CBD could have their dose decreased to 125 mg CBD Q12H (±2 hours); total daily dose 250 mg CBD. Patients taking CBD 375 mg Q12H (±2 hours); total daily dose 750 mg CBD dose could have their dose decreased to 250 mg CBD Q12H (±2 hours); total daily dose 500 mg CBD. Patients taking CBD 500 mg Q12H (±2 hours); total daily dose 1000 mg CBD dose could have their dose decreased to CBD 375 mg Q12H (±2 hours); total daily dose 750 mg or 250 mg CBD Q12H (±2 hours); total daily dose 500 mg CBD. Patients whose weight changed during the course of the study could have their dose increased or decreased.

A taper period ranging from one to three weeks, depending on the patient's dose at the time of the discontinuation, was completed. Following taper, patients were also required to complete a 4-week telephone follow-up period.

### Criteria for Evaluation

Safety Assessments: Safety assessments included collection of AEs, physical and neurological examinations, vital signs, electrocardiogram (ECG), skin check examination (investigator) and diary (parent/caregiver), and laboratory tests.

### End Points

The seizure frequency efficacy assessment end points were the median percent change from baseline in the mean monthly (28 day) frequency of seizures (SF28) over 26 weeks (Period A) for the following types, in total (“countable seizures”):
Focal impaired awareness seizures (FIAS)
Tonic-clonic seizures (TCS), including:
   Generalized tonic-clonic seizures (GTCS)
   Focal to bilateral tonic-clonic seizures (FBTCS)
The number of patients meeting 35% and 50% reduction in FIAS and TCS were recorded.

### QoL Efficacy End Points:

Change from baseline to the end of Period A in the ELDQOL
Change from baseline in “good day/bad day” assessment:

### Sleep Efficacy End Points:

Change from baseline to the end of Period A in the SDSC

### Qualitative Caregiver End Points:

Qualitative statements of parents and caregivers at the end of Period A

Analysis populations: The safety analysis set included all patients who received ≥1 dose of study drug. Patients who received ≥80 days of study drug and completed ≥80% of seizure diaries were included in the efficacy analysis and identified as the modified intent-to-treat (mITT) population.

### Results

The data indicates that ZYN-002 reduced seizure frequency in many types of difficult to treat developmental and epileptic encephalopathies, including refractory seizure types such as focal impaired awareness seizures (FIAS), generalized tonic-clonic seizures (GTCS), or focal to bilateral tonic-clonic seizures (FBTCS).

These results indicate meaningful reductions in seizures and improvements in many of the difficult behaviors and symptoms, such as sleep-related impairments, seizure intensity, fatigue, social isolation, poor cognition, and language deficits. For examples, patients experienced an improvement in sleep-related impairment including improvements in sleep quality, sleep onset, total sleep, initiating and maintain sleep, sleep wake transition, or disorders of arousal and nightmares.

Of the 48 patients enrolled in BELIEVE (ZYN2-CL-025), 40 patients completed Period A and 28 completed through Month 12 of Period B (Table 5). Only one subject withdrew consent during Period B.

TABLE 5
Patient Disposition in ZYN2-CL-025

| Status | No. of patients |
|---|---|
| Entered Period A | 48 |
| Recorded FIAS and/or TCS in Baseline | 33 |
| Completed Period A | 40 |
| Entered Period B | 29 |
| Recorded FIAS and/or TCS in Baseline | 19 |
| Withdrew consent at Week 42 | 1 |
| Completed Period B | 28 |

### Baseline Characteristics and Seizure Frequency

Of the 48 enrolled patients in BELIEVE and included in the safety analysis set, the mean age was 10.5 years (See Table 6). One quarter of the patients had Lennox-Gastaut syndrome (LGS) or Dravet syndrome.

Clinically important comorbid conditions were present in all patients and included gait and movement disorders (45.8%), sleep disturbances (39.6%), chronic respiratory conditions/infections (37.5%), ASD (29.2%), and percutaneous endoscopic gastrostomy (14.6%).

The mITT population comprised 46 patients (2 patients were excluded from efficacy analysis because they did not complete 80% of diaries or use study medication for 80 days). 33 patients had FIAS and/or TCS at baseline and constituted the population in which the seizure frequency efficacy assessment end point was measured.

TABLE 6
Baseline Demographics and Disease Characteristics,
Safety Analysis Set

| | Safety Analysis Set | |
|---|---|---|
| Demographic or Disease Characteristic | (N = 48) | |
| Age, years | | |
| Mean (range) | 10.5 | (3, 16) |
| Sex, n (%) | | |
| Male | 26 | (54.2) |
| Female | 22 | (45.8) |
| Body mass index, kg/m² | | |
| Mean (SD) | 19.2 | (4.9) |
| Seizure type^{a, b} n (%) | | |
| Focal impaired awareness | 26 | (54.2) |
| Tonic-clonic | 21 | (43.8) |
| Generalized tonic-clonic | 14 | (29.2) |
| Focal to bilateral tonic-clonic | 7 | (14.6) |
| Syndrome, n (%) | | |
| Dravet Syndrome | 8 | (16.7) |
| Lennox-Gastaut Syndrome | 5 | (10.4) |
| Epilepsy with myoclonic-atonic seziures | 6 | (12.5) |
| West Syndrome | 3 | (6.3) |
| Other^{c} | 26 | (54.2) |
| Monthly frequency of focal impaired awareness | | |
| and/or tonic-clonic seizures^{a} | | |
| median (range) | 8.2 | (0, 713) |
| Autism spectrum disorder^{d, e} n (%) | 14 | (29.2) |
| Seizure type in ASD patients, n (%) | | |
| Focal impaired awareness | 6 | (42.9) |
| Tonic-clonic | 8 | (57.1) |
| Generalized tonic-clonic | 5 | (35.7) |
| Focal to bilateral tonic-clonic | 3 | (21.4) |
| Number of concomitant ASMs, mean | 2.7 | |
| Concomitant ASMs, n (%) | 48 | (100) |
| Sodium valproate | 34 | (70.8) |
| Clobazam | 25 | (52.1) |
| Levetiracetam | 17 | (35.4) |
| Lamotrigine | 16 | (33.3) |
| Topiramate | 13 | (27.1) |

ASD: autism spectrum disorder; ASM: antiseizure medication.
^{a}During the 4-week baseline period.
^{b}For seizure type, N = 33. Thirty-three patients with focal impaired awareness and/or tonic-clonic seizures; patients could have more than one seizure type.
^{c}Includes generalized epileptic encephalopathy, focal DEE, Multifocal DEE, DEE unclassified.
^{d}ASD diagnosis per investigator.
^{e}For seizure type, N = 11. Eleven patients with focal impaired awareness and/or tonic-clonic seizures; patients could have more than one seizure type.

### Seizure Frequency Efficacy Assessment End Points

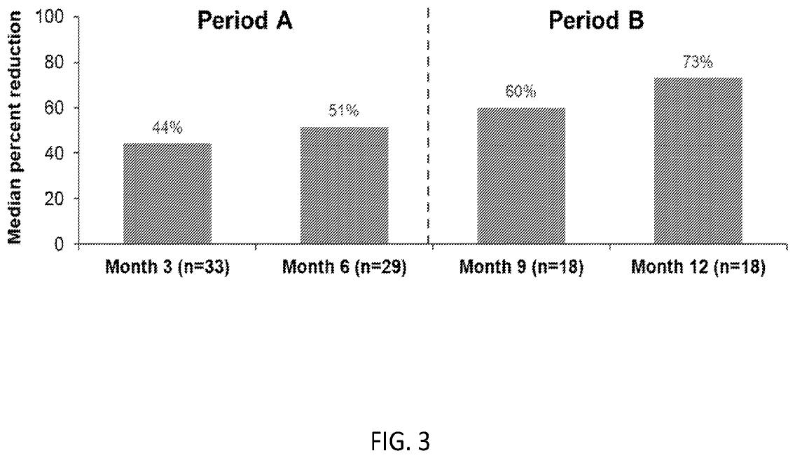

FIG. 3 is a graph indicating the efficacy of the treatment period. Specifically, FIG. 3 displays the median percentage reduction from baseline in 28-day frequency of FIAS and TCS by time point, for the patients with FIAS and/or TCS at baseline. Over the 12-month treatment period, the median percentage reduction from baseline in monthly frequency of FIAS and TCS ranged from 44% at Month 3 to 73% at Month 12.

When analyzed by seizure type, median reductions from baseline at Month 6 for FIAS, GTCS, and FBTCS were 45%, 60%, and 59%, respectively. At Month 12, the median reductions for FIAS, GTCS and FBTCS were 100%, 83% and 59% respectively.

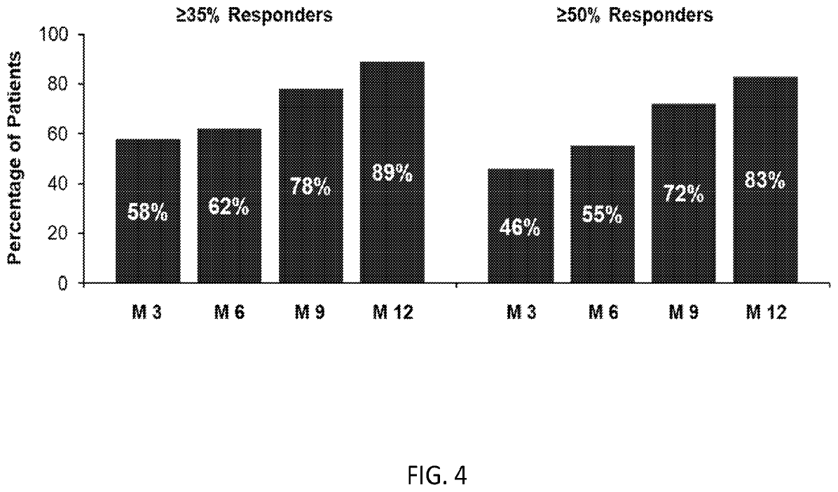

FIG. 4 is a graph indicating the percentage of patients with reduction in seizures. Specifically, FIG. 4 displays the percentage of patients with 35% and 50% reduction in FIAS and TCS by time point, for patients with FIAS and/or TCS at baseline. A substantial percentage of patients achieved ≥35% and ≥50% reduction in FIAS and TCS by Month 3 that continued through Month 12. For example, the percentage of patients achieving ≥35% increased from 58% at Month 3 to 89% at Month 12; similarly, the percentage of patients achieving ≥50% increased from 46% at Month 3 to 83% at Month 12.

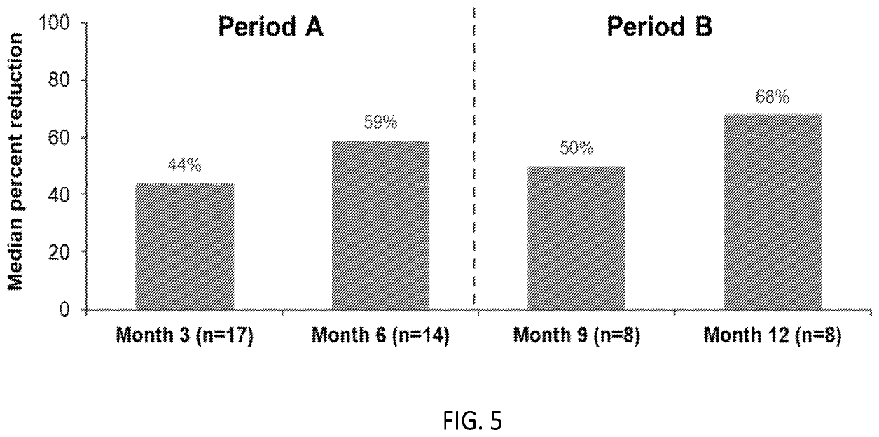

FIG. 5 is a graph indicating median percentage reduction from baseline in 28-day frequency of seizures. Specifically, FIG. 5 displays the median percentage reduction from baseline in 28-day frequency of FIAS and TCS by time point, for patients with co-morbid ASD at baseline. In patients with co-morbid ASD, median percentage reductions from baseline in monthly frequency of FIAS and TCS ranged from 44% at Month 3 to 68% at Month 12 over the 12-month treatment period.

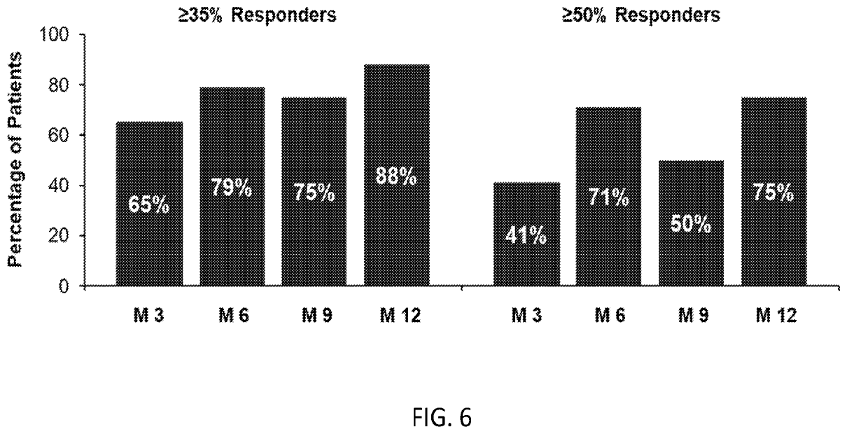

FIG. 6 is a graph indicating the percentage of patients with reduction in seizures with co-morbid autism spectrum disorder (ASD) at baseline. Specifically, FIG. 6 displays the percentage of patients with 35% and 50% reduction in FIAS and TCS by time point, for patients with co-morbid ASD at baseline. The percentage of patients achieving ≥35% and ≥50% reduction in FIAS and TCS increased over time. For example, the percentage of patients achieving ≥35% increased from 65% at Month 3 to 88% at Month 12; similarly, the percentage of patients achieving ≥50% increased from 41% at Month 3 to 75% at Month 12. Eight-out-of-nine ( 8/9) parents/caregivers provided a statement about improvement and one-out-of-nine parents/caregivers ( 1/9) stated no benefit. Reported improvements by caregivers included concentration, engagement, alertness and less sleepiness at school.

### ELDQOL

Statistically significant reductions from baseline in mean ELDQOL subscale scores for seizure severity, behavior, and mood were observed at week 26 (Table 7).

TABLE 7
Change from Baseline in Mean ELDQOL Subscale
Scores, mITT Population (n = 40)^{a}

| ELDQOL subscale | Mean (SD) | | Change^{b} | P value |
|---|---|---|---|---|
| Seizure severity | | | | |
| Baseline (n = 40) | 26 | (54.2) | | |
| Week 26 (n = 40) | 22 | (45.8) | −0.19 | 0.008 |
| Behavior | | | | |
| Baseline (n = 40) | 8 | (16.7) | | |
| Week 26 (n = 40) | 5 | (10.4) | −0.21 | 0.001 |
| Mood | | | | |
| Baseline (n = 40) | 26 | (54.2) | | |
| Week 26 (n = 40) | 21 | (43.8) | −0.15 | 0.001 |

^{a}Table 7 includes patients who completed both baseline and week 26 ELDQOL assessments;
6 patients completed the baseline assessment but did not complete the week 26 assessment.
^{b}Negative change from baseline reflects an improvement.

### Sleep Scores

Sleep disturbances effect many patients having DEE, ASD, or both DEE-ASD. Sleep disturbances include, but are not limited to, Disorders of Initiating or Maintaining Sleep (DIMS), Sleep Breathing Disorders (SBD), Disorder of Arousal/Nightmares (DA), Sleep Wake Transition Disorder (SWTD), Disorders of Excessive Somnlolence (DOES), and Sleep Hyperhidrosis. Disorders of Initiating or Maintaining Sleep (DIMS) are disorders in which the patients have difficulty falling asleep or staying asleep (e.g., insomnia). Sleep Breathing Disorders (SBD) are disorders that involve difficulty breathing during sleep (e.g., obstructive sleep apnea). Disorder of Arousal/Nightmares (DA) are mental or motor events that occur during non-REM sleep. Typical DAs include sleep walking and nightmares. In most instances, DA events are not remembered by the patient upon full awakening. Sleep Wake Transition Disorder (SWTD) include events that occur during the transition from sleeping to waking and can include rhythmic movement disorder, sleep talking, and leg cramps. Disorders of Excessive Somnlolence (DOES) are characterized by sleepiness (hypersomnolence) during normal waking hours. Sleep Hyperhidrosis is characterized by excessive sweating when falling asleep or during the night.

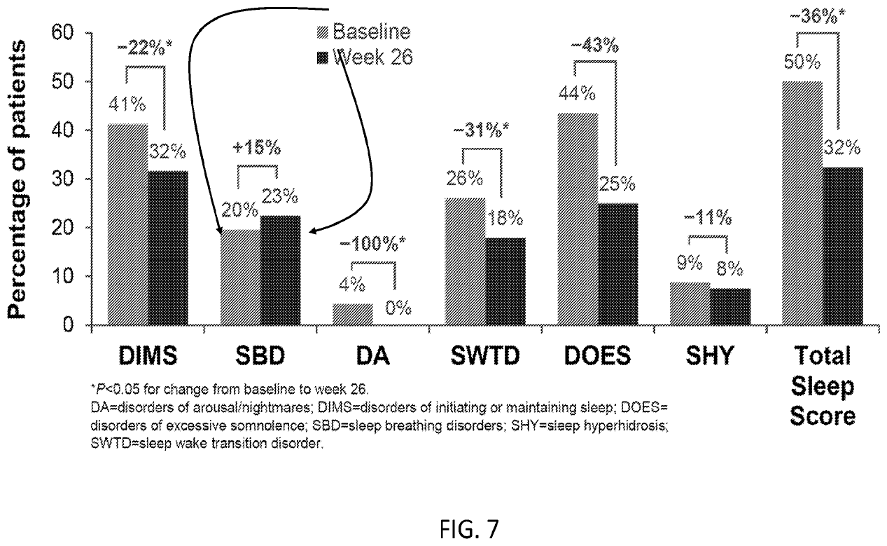

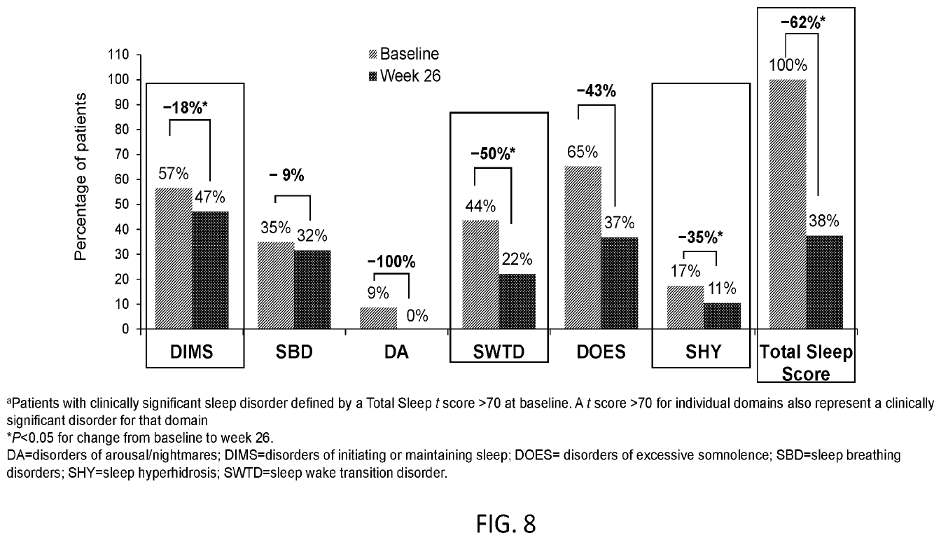

FIG. 7 displays the percentage of all patients above threshold for clinically significant sleep problems at Baseline and Week 26 of ZYN002 treatment based on the Sleep Disturbance Scale for Children (SDSC). For FIG. 7, the left bar is the Baseline percentage of patients, and the right bar is the Week 26 percentage of patients (See also Table 8). FIG. 8 shows the percentage of patients with a threshold t-score >70 at Baseline and Week 26, corresponding to clinically significant sleep problems. For FIG. 8, the left bar is the Baseline percentage of patients, and the right bar is the Week 26 percentage of patients. Statistically significant improvements from baseline in sleep scores were observed in the total score, Disorders of Initiating or Maintaining Sleep (DIMS), Disorder of Arousal/Nightmares (DA), and Sleep Wake Transition Disorder (SWTD) (See also Table 9).

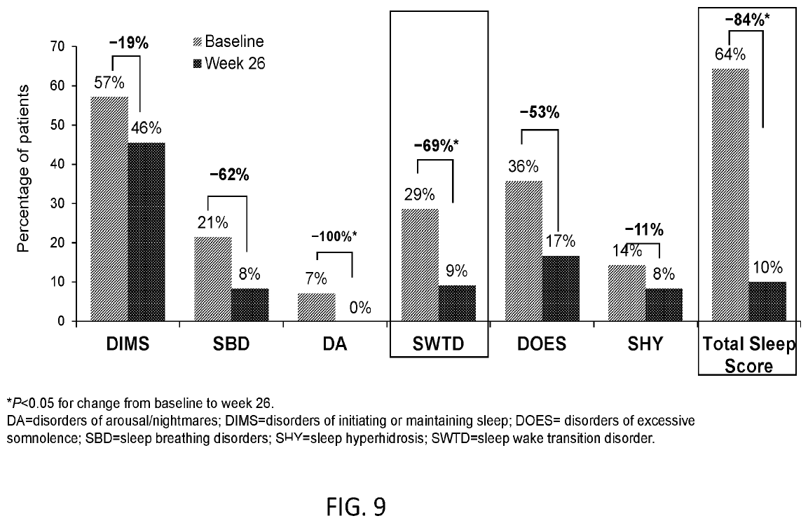

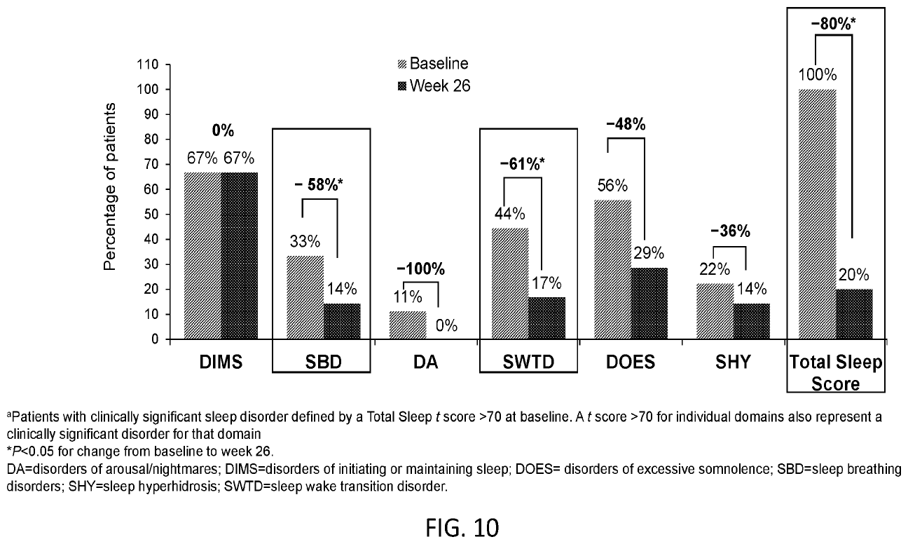

FIG. 9 displays the percentage of patients (DEE) with co-morbid ASD above threshold for clinically significant sleep problems at Baseline and Week 26 of ZYN002 treatment based on the Sleep Disturbance Scale for Children (SDSC). For FIG. 9, the left bar is the Baseline percentage of patients, and the right bar is the Week 26 percentage of patients. (See also Table 10). FIG. 10 shows the percentage of patients (DEE) with co-morbid ASD with a threshold t-score >70 at Baseline and Week 26, corresponding to clinically significant sleep problems. For FIG. 10, the left bar is the Baseline percentage of patients, and the right bar is the Week 26 percentage of patients. Statistically significant improvements from baseline in sleep scores were observed in the total score, Disorders of Initiating or Maintaining Sleep (DIMS), Disorder of Arousal/Nightmares (DA), and Sleep Wake Transition Disorder (SWTD) (See also Table 11).

TABLE 8
Change from Baseline in the SDSC - All Patients (DEE)

| | t-Score | | | |
|---|---|---|---|---|
| SDSC Factors | Mean (SD) | | Change^{a} | P value |
| Total Score | | | | |
| Baseline (n = 46) | 71.6 | (12.68) | | |
| Week 26 (n = 37) | 63.9 | (13.40) | −5.1 | 0.012 |

Disorders of Initiating and Maintaining Sleep (DIMS)

| | | | | |
|---|---|---|---|---|
| Baseline (n = 46) | 69.6 | (14.67) | | |
| Week 26 (n = 38) | 63.2 | (15.76) | −5.1 | 0.006 |

Sleep Breathing Disorders (SBD)

| | | | | |
|---|---|---|---|---|
| Baseline (n = 46) | 60.6 | (15.46) | | |
| Week 26 (n = 40) | 58.9 | (15.08) | 0.40 | 0.797 |

Disorders of Arousal/Nightmares (DA)

| | | | | |
|---|---|---|---|---|
| Baseline (n = 46) | 51.5 | (9.91) | | |
| Week 26 (n = 39) | 49.0 | (5.07) | −1.7 | 0.031 |

Sleep Wake Transition Disorder (SWTD)

| | | | | |
|---|---|---|---|---|
| Baseline (n = 46) | 65.0 | (13.09) | | |
| Week 26 (n = 39) | 60.2 | (13.78) | −4.6 | 0.030 |

Disorders of Excessive Somnolence (DOES)

| | | | | |
|---|---|---|---|---|
| Baseline (n = 46) | 68.5 | (16.76) | | |
| Week 26 (n = 40) | 63.1 | (13.82) | −3.6 | 0.100 |

Sleep Hyperhidrosis (SHY)

| | | | | |
|---|---|---|---|---|
| Baseline (n = 46) | 52.7 | (12.28) | | |
| Week 26 (n = 40) | 50.6 | (9.95) | −2.8 | 0.154 |

^{a}Negative change from baseline reflects an improvement. Change value is based on a revised Baseline mean determined from patients that completed the study. Patients that did not complete this study were excluded from the revised Baseline mean determination.

TABLE 9
Change from Baseline in the SDSC -DEE Patients >70 Total Score

| | t-Score | | | |
|---|---|---|---|---|
| SDSC Factors | Mean (SD) | | Change^{a} | P value |
| Total Score | | | | |
| Baseline (n = 23) | 81.8 | (8.7) | | |
| Week 26 (n = 16) | 70.3 | (12.0) | −8.9 | 0.01 |

Disorders of Initiating and Maintaining Sleep (DIMS)

| | | | | |
|---|---|---|---|---|
| Baseline (n = 23) | 75.7 | (15.67) | | |
| Week 26 (n = 17) | 67.4 | (17.01) | −7.3 | 0.018 |

Sleep Breathing Disorders (SBD)

| | | | | |
|---|---|---|---|---|
| Baseline (n = 23) | 66.6 | (18.73) | | |
| Week 26 (n = 19) | 63.5 | (16.92) | 0 | 1.0 |

Disorders of Arousal/Nightmares (DA)

| | | | | |
|---|---|---|---|---|
| Baseline (n = 23) | 53.2 | (13.06) | | |
| Week 26 (n = 18) | 50.1 | (6.50) | −1.9 | 0.182 |

Sleep Wake Transition Disorder (SWTD)

| | | | | |
|---|---|---|---|---|
| Baseline (n = 23) | 72.7 | (11.88) | | |
| Week 26 (n = 18) | 63.9 | (14.81) | −9.2 | 0.018 |

Disorders of Excessive Somnolence (DOES)

| | | | | |
|---|---|---|---|---|
| Baseline (n = 23) | 76.7 | (16.09) | | |
| Week 26 (n = 19) | 69.5 | (13.10) | −3.9 | 0.227 |

Sleep Hyperhidrosis (SHY)

| | | | | |
|---|---|---|---|---|
| Baseline (n = 23) | 57.3 | (15.33) | | |
| Week 26 (n = 19) | 52.5 | (10.67) | −7.2 | 0.04 |

^{a}Negative change from baseline reflects an improvement. Change value is based on a revised Baseline mean determined from patients that completed the study. Patients that did not complete this study were excluded from the revised Baseline mean determination.

TABLE 10
Change from Baseline in the SDSC -DEE-ASD Patients

| | t-Score | | | |
|---|---|---|---|---|
| SDSC Factors | Mean (SD) | | Change^{a} | P value |
| Total Score | | | | |
| Baseline (n = 14) | 75.1 | (14.77) | | |
| Week 26 (n = 10) | 62.0 | (14.77) | −8.2 | 0.003 |

Disorders of Initiating and Maintaining Sleep (DIMS)

| | | | | |
|---|---|---|---|---|
| Baseline (n = 14) | 75.8 | (18.01) | | |
| Week 26 (n = 11) | 70.6 | (16.91) | −4.9 | 0.102 |

Sleep Breathing Disorders (SBD)

| | | | | |
|---|---|---|---|---|
| Baseline (n = 14) | 62.4 | (18.00) | | |
| Week 26 (n = 12) | 56.3 | (12.45) | −3.9 | 0.072 |

Disorders of Arousal/Nightmares (DA)

| | | | | |
|---|---|---|---|---|
| Baseline (n = 14) | 53.1 | (14.26) | | |
| Week 26 (n = 11) | 49.0 | (4.5) | −1.0 | 0.341 |

Sleep Wake Transition Disorder (SWTD)

| | | | | |
|---|---|---|---|---|
| Baseline (n = 14) | 66.0 | (14.85) | | |
| Week 26 (n = 11) | 54.2 | (10.25) | −10.6 | 0.003 |

Disorders of Excessive Somnolence (DOES)

| | | | | |
|---|---|---|---|---|
| Baseline (n = 14) | 70.1 | (19.73) | | |
| Week 26 (n = 12) | 60.8 | (15.01) | −4.9 | 0.25 |

Sleep Hyperhidrosis (SHY)

| | | | | |
|---|---|---|---|---|
| Baseline (n = 14) | 51.7 | (11.87) | | |
| Week 26 (n = 12) | 48.9 | (8.72) | −3.9 | 0.084 |

^{a}Negative change from baseline reflects an improvement. Change value is based on a revised Baseline mean determined from patients that completed the study. Patients that did not complete this study were excluded from the revised Baseline mean determination.

TABLE 11
Change from Baseline in the SDSC
-DEE-ASD Patients >70 Total Score

| | t-Score | | | |
|---|---|---|---|---|
| SDSC Factors | Mean (SD) | | Change^{a} | P value |
| Total Score | | | | |
| Baseline (n = 9) | 83.9 | (10.54) | | |
| Week 26 (n = 5) | 70.6 | (16.76) | −10.4 | 0.024 |

Disorders of Initiating and Maintaining Sleep (DIMS)

| | | | | |
|---|---|---|---|---|
| Baseline (n = 9) | 81.7 | (19.39) | | |
| Week 26 (n = 6) | 79.5 | (16.18) | −4.7 | 0.37 |

Sleep Breathing Disorders (SBD)

| | | | | |
|---|---|---|---|---|
| Baseline (n = 9) | 68.3 | (19.58) | | |
| Week 26 (n = 7) | 59.4 | (15.55) | −6.7 | 0.018 |

Disorders of Arousal/Nightmares (DA)

| | | | | |
|---|---|---|---|---|
| Baseline (n = 9) | 54.1 | (17.59) | | |
| Week 26 (n = 6) | 48.8 | (4.49) | −0.0 | NA |

Sleep Wake Transition Disorder (SWTD)

| | | | | |
|---|---|---|---|---|
| Baseline (n = 9) | 72.2 | (15.06) | | |
| Week 26 (n = 6) | 57.3 | (12.83) | −15.8 | 0.006 |

Disorders of Excessive Somnolence (DOES)

| | | | | |
|---|---|---|---|---|
| Baseline (n = 9) | 77.1 | (20.73) | | |
| Week 26 (n = 7) | 68.3 | (15.10) | −3.4 | 0.544 |

Sleep Hyperhidrosis (SHY)

| | | | | |
|---|---|---|---|---|
| Baseline (n = 9) | 54.8 | (13.99) | | |
| Week 26 (n = 7) | 51.7 | (10.84) | −5.9 | 0.130 |

^{a}Negative change from baseline reflects an improvement. Change value is based on a revised Baseline mean determined from patients that completed the study. Patients that did not complete this study were excluded from the revised Baseline mean determination.

### Good Day/Bad Day Assessment

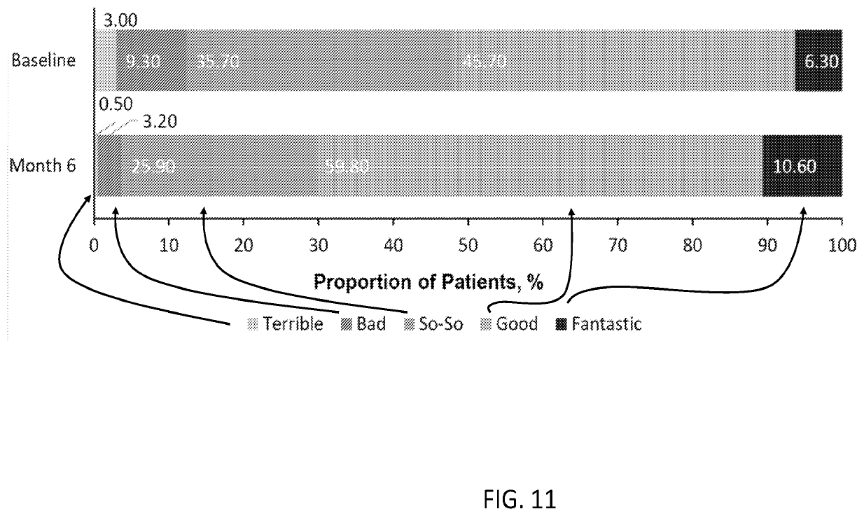

FIG. 11 displays a distribution of Good Day/Bad Day ratings at Baseline and Month 6 of ZYN002 treatment. For FIG. 11, the Baseline proportion of patients was 3.00% terrible, 9.30% terrible, 35.70% so-so, 45.70% good, and 6.30% fantastic. The Month 6 proportion of patients was 0.50% terrible, 3.20% bad, 25.90% so-so, 59.80% good, and 10.60% fantastic. In comparing Baseline to Month 6, the combined proportion of “good day” and “fantastic day” reports increased from 52.0% at baseline to 70.4%, and the combined proportion of “terrible day” and “bad day” reports decreased from 12.3% at baseline to 3.7% (FIG. 11).

### Qualitative Caregiver Feedback

The qualitative caregiver assessment was administered to parents/caregivers for 43 of the 46 patients in the mITTpopulation. 84% (n=36) of parents/caregivers provided ≥1 statement about improvement and 60% (n=26) provided ≥1 statement about worsening (Table 8). Improvement in summary measures of qualitative assessments was observed in most patients for most measures:
Any improvement: 84% (n=36)
Improved vitality: 58% (n=25)
Improvement in seizures: 51% (n=22)
Improved cognition/concentration: 47% (n=20)
Improved socially avoidant behaviors: 44% (n=19)
Improvement in irritability: 33% (n=14)
School improvement: 28% (n=12)
Medical improvement: 14% (n=6)

Table 12 displays the most frequent positive and negative qualitative statements (n≥8) made by parents and caregivers during Period A, mITT Population (N=43).

TABLE 12
Most Frequent Positive and Negative Qualitative Statements
(n ≥ 8) Made by Parents and Caregivers During Period
A, mITT Population (N = 43)

| | n (%) | |
|---|---|---|
| Improvement/Positive Statement | | |
| Behavior, Cognition, Mood | | |
| Improved alertness | 17 | (40) |
| Improved engagement/participation | 15 | (35) |
| Improved cognition | 14 | (33) |
| Attending school on time/more often | 12 | (28) |
| More energy/less fatigue | 12 | (28) |
| Improved concentration | 10 | (23) |
| Improved behavior | 9 | (21) |
| Improved mood | 8 | (19) |
| Seizures | | |
| Reduced seizure frequency | 16 | (37) |
| Reduced seizure amplitude/intensity | 9 | (21) |
| Reduced seizure duration | 9 | (21) |
| Worsening/Negative Statement | | |
| Other | | |
| Difficulty in applying gel | 11 | (26) |
| Redness, dry skin, or sensitive skin at gel application site | 8 | (19) |

The improvements seen by caregivers may be related to both improved night time sleep (e.g. sleep transitions and sleep maintenance) as well as with better daytime alertness because of better night time sleep. It is believed that daytime alertness is a function of both better night time sleep and the daytime pharmacological effects of CBD. CBD is preferentially administered in the morning or early afternoon, since it may have a stimulant effect when first administered. The combination of better sleep and the stimulant effects of CBD were shown to lead to better daytime function, better daytime cognition, and better educational performance, as can be seen from the caregiver feedback in FIG. 11 and Table 12. It is believed that both “better” days, as well as other quality of life improvements, are associated or caused by improved sleep, including improved symptoms of sleep disordered breathing.

### Safety

ZYN-002 was well tolerated in the BELIEVE clinical trial of ZYN002. Most treatment-emergent adverse events (TEAEs) (any event, whether unrelated or related to study drug) were mild or moderate. There were 30 serious adverse events reported by 14 patients over the 72-week treatment period, of which two (lower respiratory tract infection and status epilepticus) were considered possibly drug related. One patient, with a history of keratosis pilaris, discontinued study medication due to an AE (intense application site erythema); dermatologic patch testing showed this was not caused by allergic contact dermatitis from ZYN002 and was likely irritant contact dermatitis complicated by a secondary bacterial infection. There were no clinically significant changes in vital signs, ECGs, or laboratory findings except for 1 patient with a benign, isolated elevation of alkaline phosphatase at week 26 (1.69×ULN) that was not considered related to study medication.

BELIEVE is the first clinical trial of ZYN002 (transdermal CBD) in DEEs. The data suggest meaningful reductions in FIAS and TCS with ZYN002 treatment through 12 months. In the subgroup of patients with ASD, ZYN002 demonstrated meaningful reductions in FIAS and TCS seizures, with most children reaching the 35% or 50% responder threshold by Month 3 and Month 6, respectively.

Treatment with ZYN002 can be associated with clinically meaningful improvements in: seizure severity, behavior, and mood; initiating and maintaining sleep, disorders of arousal/nightmares, sleep wake transition, and overall sleep; and vitality, cognition/concentration, and socially avoidant behavior.

ZYN002 was well tolerated over 18 months of treatment in medically fragile patients of children and adolescents with DEEs. There is a positive benefit/risk profile of ZYN002 as demonstrated in this BELIEVE clinical trial in patients with DEEs and FIAS and TCS.

### Sleep Apnea

While studying the effects of CBD on sleep disorders in DEE and DEE-ADS patients, it was noted that there is a significant difference in Sleep Breathing Disorders between patients that have DEE and patients that have DEE with co-morbid ASD. Referring to Table 8, which includes combined data from patients having DEE and patients having DEE-ASD, it can be seen that there is no significant change in SBD for the combined groups. However, in the data for the DEE-ASD group (Table 10), which removes the patients having DEE without ASD, there is a substantial decrease in SBD (−3.9, p=0.072). This trend is also seen in the patients having a Total SDSC score greater than 70. In Table 9 (All Patients with DEE) there is no significant change in SBD with respect to baseline. In Table 11, which presents data from DEE-ASD patients only, the DEE-ASD patients have a decrease in SBD of −6.7 (p=0.018).

Without being bound to any theory, it is believed that this change is associated with physiological differences between DEE patients and DEE-ASD patients. Typically, DEE patients have relatively low body weight/body mass index (BMI) in comparison to the general population. In contrast, the DEE-ASD patients, and ASD patients in general, have significantly higher body weight/BMI, when compared with DEE patients. (Hill et al., Obesity and Autism, Pediatrics, 2015 December; 136(6): 1051-1061). This leads us to believe that CBD is ameliorating obstructive sleep apnea. It is also believed that sleep apnea, in general, can be treated with CBD, regardless of whether there are any other underlying causes of the sleep disorder (such as DEE and/or ADS).

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method of treating sleep disturbances in a human subject, the method comprising:
administering an effective amount of cannabidiol (CBD) to the human in need thereof to treat the sleep disturbances.

2. The method of claim 1, wherein the sleep disturbances are caused by episodes of sleep apnea.

3. The method according to claim 1 or 2, wherein the CBD is administered transdermally.

4. The method according to any of the preceding claims, wherein the effective amount of CBD ranges from about 250 mg to about 1000 mg total daily.

5. The method of any of the preceding claims, wherein the effective dose of CBD is in the range of 10-25 mg/kg per dose.

6. The method according to any of the preceding claims, wherein the CBD is administered in a single daily dose.

7. The method according to any of the preceding claims, wherein the CBD is administered in two daily doses.

8. The method according to any of the preceding claims, wherein the treatment includes an improvement in sleep-related impairment.

9. The method of claim 8, wherein an improvement in sleep-related impairment includes improvements in sleep quality, sleep onset, total sleep, initiating and maintain sleep, sleep wake transition, or disorders of arousal and nightmares.

10. The method according to any of the preceding claims, wherein the CBD is a pure CBD.

11. The method according to any of the preceding claims, wherein the CBD is a synthetic CBD.

12. The method according to any of claims 1-9, wherein the CBD is botanically derived.

13. The method according to any of the preceding claims, wherein the cannabidiol is (−)-cannabidiol.

14. The method according to any of the preceding claims, wherein the CBD is formulated as a gel.

15. The method of claim 14, wherein the CBD is formulated as a permeation-enhanced gel.

16. The method of any one of the preceding claims, wherein the subject suffers from obstructive sleep apnea.

17. The method of claim 16, wherein the obstructive sleep apnea is associated with obesity or an overweight condition of the subject.

18. The method according to any of the preceding claims, wherein the subject is an adult.

19. The method according to any of claims 1-17, wherein the subject is between 3 and 18 years old.

20. The method according to any of the preceding claims, wherein the subject has ASD.

21. The method according to any of the preceding claims, wherein the subject has DEE-ASD.

22. The method according to any of the preceding claims, wherein the subject has a BMI of greater than 25.

23. The method according to any of the preceding claims, wherein, upon beginning administering the effective amount of CBD, circadian rhythm is maintained or improved.
